(19) United States
(12) Patent Application Publication
(10) Pub. No.: US 2003/0049597 A1
(43) Pub. Date: Mar. 13, 2003

(54) Chimeric fluorescent enzymes and uses thereof

(76) Inventors: Sanford M. Simon, New York, NY (US); Yu Chen, New York, NY (US)

Correspondence Address: KLAUBER & JACKSON, 411 HACKENSACK AVENUE, HACKENSACK, NJ, 07601

(21) Appl. No.: 09/797,496

(22) Filed: Mar. 1, 2001

(51) Int. Cl. C12Q 1/00; C12Q 1/37; C12N 15/87

(52) U.S. Cl. 435/4; 435/23; 435/455

(57) ABSTRACT

Method for similtaneously quantifying in situ the relationship between an enzyme and its substrate, especially by the use of fluorescence.

The research leading to the present invention was supported, at least in part, by the National Institutes of Health Grant R01CA81257 NIH MSTP Grant GM07739. Accordingly, the Government may have certain rights in the invention.

### BACKGROUND

This invention relates to the field of monitoring fluorescent-tagged enzymes and their reactions in living cells. Of particular interest are the processing of fluorescent enzymatic substrates, especially chemotherapeutic drugs.

In recent years there has been an explosion in the ability to do biochemistry in living cells. One major contributing factor has been the use of the Aequorea victoria green fluorescent protein (GFP) which is endogenously fluorescent (Chalfie et al., 1994; U.S. Pat. No. 5,491,084 of M. Chalfie et al.). GFP has allowed many different proteins and cellular processes to be examined in situ, that is, in the living cell. In short, the coding region for the green-fluorescent protein is conjugated to coding region of the protein of interest. Often, when GFP is conjugated to either the amino or carboxy terminus of the protein, the activity of the protein is unaffected. The GFP is relatively insensitive to photo bleaching and appears not to cause any cellular toxicity. GFP has been conjugated to many hundreds of different proteins. At least four different companies are selling commercial forms of GFP that have been optimized for either expression, fluorescence intensity or fluorescent emission. Variants of GFP have been generated that are either sensitive to pH or with different emission spectra.

It would be useful to be able to use the GFP protein linked to enzymes. In such a case, the barrier to success is higher as the protein must not affect the enzymatic activity of enzyme. Nevertheless such fluorescing enzymes would be useful for the study of a large number of processes. One such process, discussed in detail here, is the phenomenon of Multidrug resistance (MDR) in chemotherapy.

MDR is the major obstacle to the successful chemotherapeutic treatment of human cancers (Simon and Schindler, 1994; Ling, 1997). Often a cancer becomes resistant to many drugs of diverse structures and mechanisms after exposure to only a few (Biedler et al., 975;Bech-Hansen et al., 1976); it is then said to have developed multidrug resistance. Such MDR cancers often overexpress P-glycoprotein (Pgp), an ATP-binding cassette (ABC) protein encoded by the MDR1 gene (Kartner et al., 1983) (for a review, see (Gottesman and Pastan, 1993;Stein, 1997;Wadkins and Roepe, 1997;Eytan and Kuchel, 1999)). These cells are characterized by the decreased accumulation of many classes of drugs, commonly called MDR drugs. The accumulation of MDR drugs in these cells is enhanced by other compounds known as MDR reversers.

Most studies of MDR have used drug-resistant cells generated by selection with chemotherapeutic drugs. These drugs are highly mutagenic and tumor cells are genetically unstable. Thus this selection process leads to a host of changes in cellular physiology that may result in drug resistance. Among them: decreased susceptibility to apoptosis (Robinson et al., 1997), increased DNA repair and drug metabolism (Deffie et al., 1988); increased cellular pH (Thiebaut et al., 1990;Roepe et al., 1993;Simon et al., 1994); decreased lysosomal and endosomal pH (Schindler et al., 1996;Altan et al., 1998); decreased plasma membrane potential (Roepe et al., 1993); increased plasma membrane conductance to chloride (Gill et al., 1992) and ATP (Abraham et al., 1993); and increased rates of vesicle transport (Altan et al., 1999). Studying a subclone after selection in chemotherapeutics has made it difficult to determine the degree to which Pgp contributes to MDR in any particular drug-selected cell line. It has also complicated studying Pgp's underlying mechanism. The most accepted mechanism is that Pgp is an ATP-dependent drug efflux pump. Alternatively, it has been proposed that Pgp raises the cytosolic pH and lowers the plasma membrane potential, thereby decreasing the accumulation of weak-bases and positively charged drugs into the cytosol (Hoffman et al., 1996).

To elucidate the mechanism of Pgp, we used a novel technique of in situ biochemistry. We transiently expressed a fusion protein between Pgp and green fluorescent protein (GFP) to produce a mixed population of cells with a broad range of expression levels. Fluorescence was used to quantify simultaneously the expression and the activity of Pgp in individual cells. This eliminates the confounding aspects of drug selection or even clonal expansion. Expression of Pgp, in the absence of drug selection, was shown to be sufficient to produce drug resistance to a spectrum of unrelated chemotherapeutic drugs. The resulting quantification of the relation between Pgp expression and activity is consistent with the concept of Pgp as an active efflux pump and not consistent with cooperativity between either Pgp, substrate or inhibitor molecules. Last, our study demonstrates the advantages of using in situ assays for quantitative biochemistry.

### SUMMARY OF THE INVENTION

A quantification process for simultaneously quantifying in situ the relationship between an enzyme and its substrate, said process comprising the steps of:

1) creating a population of cells that synthesize said enzyme, such that all cells in the population do not contain equal amounts of said enzyme, and wherein the enzyme is tagged with a fluorescent agent;

2) incubating the population of cells in the presence of the substrate;

3) simultaneously, by optical means, quantifying in each member of the population the intracellular concentration of the enzyme and its enzymatic activity.

It is understood that the population to which the process is applied may be a sub-population of a larger population of cells.

Preferred embodiments of the quantification include but are not limited to, those wherein one or more of the following features apply:

(1) the enzyme is fluorescently tagged, especially by fusing it to a fluorescent protein so that the enzyme and the fluorescent protein are part of the same protein;

(2) the substrate has a color or, more preferably, is fluorescent; which color or fluorescence is distinguishable from the emitted fluorescence of the tagged enzyme;

(3) a product of the enzyme's action on the substrate has a color or, more preferably, is fluorescent, which color or fluorescence is distinguishable from the emitted fluorescence of the tagged enzyme;

(4) the population of cells is created by transfecting it with a nucleic acid construct that, upon infecting a cell, causes expression of the fluorescently tagged enzyme, preferably by virtue of the fact that the construct comprises a base sequence coding for said enzyme;

(5) quantification of fluorescence comprises analysis of individual cells; and

(6) step (3) of the process is performed a plurality of times.

Scenarios for implementation, the invention include, but are not limited to:

1) quantifying the role of an enzyme in the intracellular uptake of a substrate (See, for example, Example 1); and

2) quantifying the role of an enzyme in the intracellular conversion of a compound A to a compound B (See, for example, Example 2).

Preferred fluorescent protein tags are GFP and fluorescent proteins derived therefrom.

An enzyme is a protein that accelerates a reaction in a manner such that the enzyme's chemical composition does not change. The acceleration is accomplished by the action of the enzyme on a substrate. The reaction may result in an alteration of the structure of the substrate (e.g. by it undergoing hydrolysis, by part of its structure being substituted with another moiety) or in the alteration of the location of the substrate within a cell or in an alteration of the location of the substrate with respect to a cell (movement of the substrate into or out of the cell.)

The enzyme in the present invention may comprise one or more polypeptide chains. It is only important that the fluorescent tag not affect the ability of the enzyme to catalyze the reaction of interest.

The present invention is useful for the simultaneous detection and/or location of an enzyme protein and its effect on a substrate in living cell, which information is important for understanding enzymatic processes at the cellular level and for chemotherapeutic processes.

It is also highly preferred to do a “background” control because any apparatus used to detect fluorescence might, in addition to the fluorescence in step (3) due to the fluorescent fusion protein and the substrate, also detect “background” fluorescence due to other factors while performing step (3).

As a result, in a preferred embodiment of the method, the fluorescence emitted by the enzymatic fluorescent fusion protein in the absence of the substrate is measured and/or the fluorescence emitted in the absence of the fluorescent fusion protein by the fluorescent substrate or its product (if the product is to be monitored) is measured.

Methods for measuring the background include (1) the use of cells that do not make the fluorescing fusion protein, but are otherwise the same as the cells in the process, and (2) the use of cells that make the fusion protein but to which no substrate has been added.

### BRIEF DESCRIPTION OF THE DRAWINGS

FIG. 1. Effect of PgpGFP expression on the accumulation of fluorescent dyes. A) PgpGFP transfected HeLa cells were incubated for 30 minutes with 1 μM daunorubicin without (top) or with (bottom) 40 μg/ml verapamil. GFP and daunorubicin fluorescence were imaged using a confocal microscope. The fluorescence profile along the line drawn in the merge image was quantified (right). (B) Cells were incubated for 30 minutes with 200 nM TMRE and 200 nM Ho342 and the fluorescence images of GFP, TMRE and Ho342 were acquired using an epifluorescence microscope. (C) Cells were incubated for 30 minutes with 5 μM Fura Red AM and GFP and Fura Red fluorescence were imaged on a confocal microscope. Scale bar is 20 μM.

FIG. 2. Effect of PgpGFP expression on vincristine mediated microtubule depolymerization. PgpGFP transfected HeLa cells were incubated with 2 μM or 80 nM vincristine for 30 minutes and stained with a Cy3-conjugated anti-b-tubulin antibody. Arrows indicate PgpGFP expressing cells and arrowheads indicate non-expressing cells. Scale bar is 20 μM.

FIG. 3. FACS analysis of TMRE accumulation as a function of PgpGFP expression-(A) Histogram of GFP fluorescence of HeLa cells transfected with PgpGFP. Approximately 50% cells were non-expressing showing a single peak of approximately 5×10⁰ units. Transfected cells showed a >100-fold range of GFP fluorescence. (B) Dot plot of transfected cells incubated for 30 minutes with 50 nM TMRE. The solid diagonal line of slope −1 was fit to data. To estimate the average TMRE fluorescence exhibited by cells with GFP fluorescence of 10³ units, a dashed line was drawn from the center of cell density at 10³ down and across to the x- and y-axis. At all levels of PgpGFP expression, there were some cells that showed TMRE fluorescence of 10² on the y-axis (marked as a horizontal oval). Co-incubation with propidium iodide selectively increased the fluorescence of these cells indicating that they were most likely dead (data not shown). (C-G) Dot plot of cells co-incubated with the indicated verapamil concentration. The solid diagonal line was reproduced from B for reference. The average TMRE fluorescence exhibited by cells with GFP fluorescence of 10³ units was estimated as in B. (H) The ratio of drug in/drug out was modeled is described in text. Plots of Dᵢₙ,Dₒᵤₜ as a function of number of Pgp per cells are shown for five different catalytic constants of 10, 1, 0.1, 0.01, and 0.001 drug molecules per pump per second. (I) Plot of average TMRE fluorescence of cells exhibiting GFP fluorescence of 10³ as a function of verapamil concentration from the dash lines drawn in B-G. Open circle indicates no verapamil control (not 1 μM).

FIG. 4. Effect of PgpGFP expression on cellular pH. PgpGFP transfected HeLa cells were loaded with SNARF-1 by a 30 minute incubation with 1 μM SNARF-1 AM. (A-F) SNARF-1 was calibrated by measuring the fluorescence cells incubated in calibration solution at pH 6.75, 7.25, 7.75 (shown) and 7.0, 7.5, 8.0 (not shown). The GFP image identifies PgpGFP expressing cells (left). The ratio of the SNARF-1 base:acid fluorescence are shown as a pseudocolor intensity image (right). The scale on the right of the lookup color bar represent the average SNARF-1 ratio calibrated at the indicated pH values. (G-H) The GFP and SNARF-1 fluorescent ratio images of cells in media. Scale bar is 20 μM.

### DETAILED DESCRIPTION OF THE INVENTION

Abbreviations and glossary

ABC stands for ATP-binding cassette;

GFP stands for green fluorescent protein;

“GFP” fusion proteins include proteins wherein one of the fused proteins is GFP, a fluorescent protein that is derived from, or is a variant of, GFP.

AM, acetoxymethyl;

Ho342 stands for Hoechst 33342;

MDR stands for multidrug resistance;

Pgp stands for P-glycoprotein;

TMRE stands for tetramethylrhodamine methyl ester.

A “plurality” means more than one.

A “coding sequence” or a “coding region” of a nucleic acid for a designated protein refers to a region in an mRNA molecule that contains the base sequence which is translated into an amino acid sequence (i.e., that encodes that amino acid sequence), it covers any DNA or RNA sequence that is complementary in base sequence to such an mRNA sequence, it covers any DNA sequence that is the same as such an mRNA sequence (except that T is used in place of U) and, in the case of a DNA sequence that alternates introns with exons so that the sequence can be processed to make an mRNA molecule, “coding sequence” or “coding region” covers the populations of coding exons that determine the amino acid coding region of the mRNA molecule (i.e., that encode that amino acid sequence).

An RNA base sequence (or molecule) is equivalent to a DNA base sequence (or molecule) if they are identical except that U in the RNA base sequence replaced T in the DNA base sequence.

The following one letter codes are used to represent amino acids:

S-serine, T-threonine, N-asparagine, Q-glutamine, K-lysine, R-arginine, H-histidine, E-glutamic acid, D-aspartic acid, C-cystine, G-glycine, P-proline, A-alanine, I-isoleucine, L-leucine, M-methionine, F-phenylalanine, W-tryptophan, V-valine, Y-tyrosine, X-any amino acid.

The following one letter codes are used to represent nucleic acids:

A-adenine, C-cytosine, G-guanine, T-thymidine, R represents A or G, Y represents T or C, N represents any nucleic acid.

GFP and Related Proteins

A highly preferred fluorescing protein is the green-fluorescing protein (GFP) of Aequorea victoria, and derivatives or variants thereof which retain GFP's ability to fluoresce. (“Derivatives” and “variants” are essentially used interchangeably herein.) Many useful details for using GFP in fusion proteins are disclosed in U.S. Pat. No. 5,491,084 of M. Chlafie et al., (“The Chalfie patent.”), which is hereby in its entirety incorporated herein by reference. The Chlafie patent discloses, inter alia, sources of information for the amino acid sequence of GFP and also that the plasmid pGFP10.1 which contains cDNA for a functional version of GFP has been deposited with ATCC accession number 75547 with the American Type Culture Collection in Rockville, Md. The base sequence of that cDNA is also incorporated herein by reference. GFP variants retain the ability to fluoresce have been described in International Application Number PCT/US99/12850 and its publication WO 99/64592.

Other fluorescing proteins include, but are not limited to, yellow fluorescent protein (YFP), red fluorescent protein (RFP), and cyan fluorescent protein (CFP), and blue fluorescent protein (BFP). CFP, BFP, and YFP are derived from GFP.

Fusion Proteins

Two fused proteins are proteins that are non-overlapping proteins of a single polypeptide sequence. Generally, the proteins are fused because their coding regions occur on the same mRNA molecule, which molecule has been generated as a result of recombinant DNA technology. Also, those two coding regions are normally not found on the same naturally occurring mRNA molecule. It is preferred that, within the coding region for the fusion protein, the coding region of the cellular enzyme is upstream relative to one coding region of the fluorescence protein. To this end, one can construct a plasmid (or other cloning vehicle) that comprises the transcriptional template for the fluorescent protein coding sequences and at least some of its downstream noncoding mRNA sequences. Just upstream of that transcriptional template is the insertion point for the cDNA of the cellular enzyme that will part of the enzymatic fusion protein.

Method for Making a Fusion Protein with the GFP of Aequorea victoria

There are many well known methods for making a DNA construct (e.g., plasmid or vector) that comprises a regulatory element (such as a promoter for RNA polymerase binding, sequences needed for activation of transcription, and sequences that when part of the RNA transcript are needed for ribosome binding and correct initiation and termination of translation), as well as the coding regions for the fusion protein. Such DNA constructs can be used to modify host cells so that they express fusion proteins of which GFP is one component.

Of the many papers published on GFP fusion proteins, examples include Clemen et al. (1999) using fibroblasts, Bevan et al. (1999) using CHO cells, Brachat et al. (2000) using yeast, and Petersson et al. (1999) using yeast.

### Materials and Methods used in Example 1

Cell Culture

HeLa cells (ATCC CCL-2) were cultured as per ATCC recommendations. MCF-7/ADR cells were cultured as described (Altan et al., 1998).

Construction and Expression of Vector

All restriction enzymes and T4 DNA ligase were from New England Biolabs (Beverly, Mass.). pGEM3Zf(−)Xba-MDR1.1, a phagemid containing the human MDR1 cDNA, was purchased from ATCC. To make the PgpGFP fusion vector, site-directed mutagenesis using the UNG-DUT method (Kunkel, 1985) was performed to eliminate the 3′ stop codon and introduce a SalI site. The Pgp open reading frame was excised using XbaI on the 5′ end and SalI on the 3′ end and inserted into plasmid pEGFP-N1 (Clontech, Palo Alto, Calif.) cut with the NheI and SalI. The PgpCFP fusion vector was subsequently constructed by replacing the coding region for EGFP with that for ECFP from pECFP (Clontech)(ECFP, is a variant of CFP). Transfections used Fugene 6 reagent (Roche Molecular Biomedical, Germany).

Transient Infection

Transfection can be achieved by any of many procedures including, but not limited to, calcium phosphate-mediated infection, lipid-based systems (e.g., Fugene, Lipofectamine) and microinjection.

Fluorescent Microscopy

Epifluorescence microscopy was done on an inverted IX-70 microscope (Olympus America, Melville, N.Y.). The image was collected using the Orca cooled CCD camera (Hamamatsu Photonics, Hamamatsu City, Japan), an IMAQ-1424 digital image acquisition card and in-house software written in LabVIEW (National Instruments, Austin, Tex.). Excitation was provided using a 150 Watt Xenon arc lamp (OptiQuip, Beaver Falls, Pa.). Excitation and emission filters were selected using filter wheels (Ludl Electronic Products, Hawthorne, N.Y.). All filters were from Chroma Technology (Brattleboro, Vt.). The following excitation and emission filters were used for epi-fluorescent microscopy: CFP: λ^{ex}, =400-430 nm, λₑₘ=460-500 nm; GFP, BCECF, Calcein, SNAFL-1, SNAFL calcein: λₑₓ=480-490 nm, λₑₘ=500-550 nm; Hoechst 342, FURA-2: λₑₓ=340-380 nm, λₑₘ=430-470 nm; tetramethylrhodamine methyl ester, SNARF-1, SNARF calcein: λₑₓ=530-560 nm, λₑₘ=570-650 nm.

Confocal microscopy was done on an upright Axiplan 2 microscope with a LSM 510 confocal attachment (Carl Zeiss, Thomwood, N.Y.). Excitation was provided an Argon/Krypton laser with lines at 488 nm and 568 nm and a Helium/Neon laser at 633 nm. The following laser lines and emission filters were used for confocal microscopy: GFP λₑₓ=488 nm, λₑₘ=500-530 nm, Texas Red, Cy3, TMRE: λₑₓ=568 nm, λₑₘ=580 nm LP, Fura Red, daunorubicin: λₑₓ=488 nm, λₑₘ=580 nm LP.

Immunofluorescence

Cells were plated on 18 mm glass coverslips placed in 12-well dishes. For Pgp surface staining, live cells were incubated with 1.22 μg/ml 4E3 (Dako, Carpinteria, Calif.), or 2 μg/ml UIC2 (Immunotech, Marseille, France), washed, stained with Texas Red conjugated anti-mouse IgG antibody at 1:500 (Sigma) and fixed. For tubulin staining, cells were incubated for 30 minutes in the presence of indicated concentrations of vincristine (Calbiochem), colchicine (Calbiochem) or nocodazole (Sigma). Cells were then fixed, permeabilized, and labeled with 2 μg/ml Cy3-labeled anti-b-tubulin antibody, clone TUB 2.1 (Sigma).

Dual Labeling with Drugs and Dyes in Living Cells

All fluorescent dyes were from Molecular Probes. Daunorubicin was from Calbiochem. Cells were incubated with the indicated drug or dye for the indicated amount of time in Opti-MEM without phenol red and with 10 mM Hepes (Life Technologies, Rockville, Md.) in a 5% CO₂, 37° C. incubator prior to observation.

Ratiometric pH Imaging

SNARF-1 is ratiometric pH indicator which emits at 640 nm in the basic form and 580 nm in the acidic form. Cells were loaded with 1 μM SNARF-1 AM in Opti-MEM for 30 minutes and resuspended in dye free Opti-MEM prior imaging. Three images were acquired for each field: GFP, SNARF-1 acid (λₑₓ=530-560 nm, λₑₘ=570-590 nm), and SNARF-1 base (λₑₓ=530-560 nm, λₑₘ=600-660 nm). Calibration was done as previously described (Altan et al., 1998).

FACS Analysis

Cells were dissociated using Cell Stripper (Cellgro, Herndon, Va.), incubated for 30 minutes in OptiMEM with 50 nM TMRE and analyzed using a FACScan and CellQuest software (Becton Dickinson, San Jose, Calif. GFP and TMRE fluorescence were acquired using FL1 (515-545 nm) and FL2 (564-606 nm) respectively. To estimate the number of GFP from fluorescence, 6 μm SPHERO yellow calibration particles (Pharmingen, San Diego, Calif.) were used. The number of GFP molecules per cell was estimated to be 1.5× the FITC equivalent fluorescence units (Tsien, 1998).

### Information Pertinent to the Use of Fluorescent Proteins

Information Regarding EGFP

This information including SEQ ID NO: 1 is based on information from the Clontech web site. pEGFP-N1 encodes a red-shifted variant of wild-type GFP optimized for fluorescence and expression in mammalian cells. (Excitation maximum=488 nm; emission maximum=507 nm.) In addition to substitutions noted below, the coding sequence of the EGFP gene contains more than 190 silent base changes which correspond to human codon-usage preferences. Genes cloned into the MCS will be expressed as fusions to the N-terminus of EGFP if they are in the same reading frame as EGFP and there are no intervening stop codons. Stably transfected eukaryotic cells can be selected using G418.

A plasmid base sequence comprising the coding sequence for EGFP is SEQ ID NO: 1 and is:

| | |
|---|---|
| TAGTTATTAA TAGTAATCAA TTACGGGGTC ATTAGTTCAT AGCCCATATA TGGAGTTCCG | (End of SEQ ID NO:1) |
| CGTTACATAA CTTACGGTAA ATGGCCCGCC TGGCTGACCG CCCAACGACC CCCGCCCATT | |
| GACGTCAATA ATGACGTATG TTCCCATAGT AACGCCAATA GGGACTTTCC ATTGACGTCA | |
| ATGGGTGGAG TATTTACGGT AAACTGCCCA CTTGGCAGTA CATCAAGTGT ATCATATGCC | |
| AAGTACGCCC CCTATTGACG TCAATGACGG TAAATGGCCC GCCTGGCATT ATGCCCAGTA | |
| CATGACCTTA TGGGACTTTC CTACTTGGCA GTACATCTAC GTATTAGTCA TCGCTATTAC | |
| CATGGTGATG CGGTTTTGGC AGTACATCAA TGGGCGTGGA TAGCGGTTTG ACTCACGGGG | |
| ATTTCCAAGT CTCCACCCCA TTGACGTCAA TGGGAGTTTG TTTTCGCACC AAAATCAACG | |
| GGACTTTCCA AAATGTCGTA ACAACTCCGC CCCATTGACG CAAATGGGCG GTAGGCGTGT | |
| ACGGTGGGAG GTCTATATAA GCAGAGCTGG TTTAGTGAAC CGTCAGATCC GCTAGCGCTA | |
| CCGGACTCAG ATCTCGAGCT CAAGCTTCGA ATTCTGCAGT CGACGGTACC GCGGGCCCGG | |
| GATCCACCGG TCGCCACCAT GGTGAGCAAG GGCGAGGAGC TGTTCACCGG GGTGGTGCCC | |
| ATCCTGGTCG AGCTGGACGG CGACGTAAAC GGCCACAAGT TCAGCGTGTC CGGCGAGGGC | |
| GAGGGCGATG CCACCTACGG CAAGCTGACC CTGAAGTTCA TCTGCACCAC CGGCAAGCTG | |
| CCCGTGCCCT GGCCCACCCT CGTGACCACC CTGACCTACG GCGTGCAGTG CTTCAGCCGC | |
| TACCCCGACC ACATGAAGCA GCACGACTTC TTCAAGTCCG CCATGCCCGA AGGCTACGTC | |
| CAGGAGCGCA CCATCTTCTT CAAGGACGAC GGCAACTACA AGACCCGCGC CCAGGTGAAG | |
| TTCGAGGGCG ACACCCTGGT GAACCGCATC GAGCTGAAGG GCATCGACTT CAAGGAGGAC | |
| GGCAACATCC TGGGGCACAA GCTGGAGTAC AACTACAACA CCCACAACGT CTATATCATG | |
| GCCGACAAGC AGAAGAACGG CATCAAGGTG AACTTCAAGA TCCGCCACAA CATCGAGGAC | |
| GGCAGCGTGC AGCTCGCCGA CCACTACCAG CAGAACACCC CCATCGGCGA CGGCCCCGTG | |
| CTGCTGCCCG ACAACCACTA CCTGAGCACC CAGTCCGCCC TGAGCAAAGA CCCCAACGAG | |
| AAGCGCGATC ACATGGTCCT GCTGGAGTTC GTGACCGCCG CCGGGATCAC TCTCGGCATG | |
| GACGAGCTGT ACAAGTAAAG CGGCCGCGAC TCTAGATCAT AATCAGCCAT ACCACATTTG | |
| TAGAGGTTTT ACTTGCTTTA AAAAACCTCC CACACCTCCC CCTGAACCTG AAACATAAAA | |
| TGAATGCAAT TGTTGTTGTT AACTTGTTTA TTGCAGCTTA TAATGGTTAC AAATAAAGCA | |
| ATAGCATCAC AAATTTCACA AATAAAGCAT TTTTTTCACT GCATTCTAGT TGTGGTTTGT | |
| CCAAACTCAT CAATGTATCT TAAGGCGTAA ATTGTAAGCG TTAATATTTT GTTAAAATTC | |
| GCGTTAAATT TTTGTTAAAT CAGCTCATTT TTTAACCAAT AGGCCGAAAT CGGCAAAATC | |
| CCTTATAAAT CAAAAGAATA GACCGAGATA GGGTTGAGTG TTGTTCCAGT TTGGAACAAG | |
| AGTCCACTAT TAAAGAACGT GGACTCCAAC GTCAAAGGGC GAAAAACCGT CTATCAGGGC | |
| GATGGCCCAC TACGTGAACC ATCACCCTAA TCAAGTTTTT TGGGGTCGAG GTGCCGTAAA | |
| GCACTAAATC GGAACCCTAA AGGGAGCCCC CGATTTAGAG CTTGACGGGG AAAGCCCGCG | |
| AACGTGGCGA GAAAGGAAGG GAAGAAAGCG AAAGGAGCGG GCGCTAGGGC GCTGGCAAGT | |
| GTAGCGGTCA CGCTGCGCGT AACCACCACA CCCGCCGCGC TTAATGCGCC GCTACAGGGC | |
| GCGTCAGGTG GCACTTTTCG GGGAAATGTG CGCGGAACCC CTATTTGTTT ATTTTTCTAA | |
| ATACATTCAA ATATGTATCC GCTCATGAGA CAATAACCCT GATAAATGCT TCAATAATAT | |
| TGAAAAAGGA AGAGTCCTGA GGCGGAAAGA ACCAGCTGTG GAATGTGTGT CAGTTAGGGT | |
| GTGGAAAGTC CCCAGGCTCC CCAGCAGGCA GAAGTATGCA AAGCATGCAT CTCAATTAGT | |
| CAGCAACCAG GTGTGGAAAG TCCCCAGGCT CCCCAGCAGG CAGAAGTATG CAAAGCATGC | |
| ATCTCAATTA GTCAGCAACC ATAGTCCCGC CCCTAACTCC GCCCATCCCG CCCCTAACTC | |
| CGCCCAGTTC CGCCCATTCT CCGCCCCATG GCTGACTAAT TTTTTTTATT TATGCAGAGG | |
| CCGAGGCCGC CTCGGCCTCT GAGCTATTCC AGAAGTAGTG AGGAGGCTTT TTTGGAGGCC | |
| TAGGCTTTTG CAAAGATCGA TCAAGAGACA GGATGAGGAT CGTTTCGCAT GATTGAACAA | |
| GATGGATTGC ACGCAGGTTC TCCGGCCGCT TGGGTGGAGA GGCTATTCGG CTATGACTGG | |
| GCACAACAGA CAATCGGCTG CTCTGATGCC GCCGTGTTCC GGCTGTCAGC GCAGGGGCGC | |
| CCGGTTCTTT TTGTCAAGAC CGACCTGTCC GGTGCCCTGA ATGAACTGCA AGACGAGGCA | |
| GCGCGGCTAT CGTGGCTGGC CACGACGGGC GTTCCTTGCG CAGCTGTGCT CGACGTTGTC | |
| ACTGAAGCGG GAAGGGACTG GCTGCTATTG GGCGAAGTGC CGGGGCAGGA TCTCCTGTCA | |
| TCTCACCTTG CTCCTGCCGA GAAAGTATCC ATCATGGCTG ATGCAATGCG GCGGCTGCAT | |
| ACGCTTGATC CGGCTACCTG CCCATTCGAC CACCAAGCGA AACATCGCAT CGAGCGAGCA | |
| CGTACTCGGA TGGAAGCCGG TCTTGTCGAT CAGGATGATC TGGACGAAGA GCATCAGGGG | |
| CTCGCGCCAG CCGAACTGTT CGCCAGGCTC AAGGCGAGCA TGCCCGACGG CGAGGATCTC | |
| GTCGTGACCC ATGGCGATGC CTGCTTGCCG AATATCATGG TGGAAAATGG CCGCTTTTCT | |
| GGATTCATCG ACTGTGGCCG GCTGGGTGTG GCGGACCGCT ATCAGGACAT AGCGTTGGCT | |
| ACCCGTGATA TTGCTGAAGA GCTTGGCGGC GAATGGGCTG ACCGCTTCCT CGTGCTTTAC | |
| GGTATCGCCG CTCCCGATTC GCAGCGCATC GCCTTCTATC GCCTTCTTGA CGAGTTCTTC | |
| TGAGCGGGAC TCTGGGGTTC GAAATGACCG ACCAAGCGAC GCCCAACCTG CCATCACGAG | |
| ATTTCGATTC CACCGCCGCC TTCTATGAAA GGTTGGGCTT CGGAATCGTT TTCCGGGACG | |
| CCGGCTGGAT GATCCTCCAG CGCGGGGATC TCATGCTGGA GTTCTTCGCC CACCCTAGGG | |
| GGAGGCTAAC TGAAACACGG AAGGAGACAA TACCGGAAGG AACCCGCGCT ATGACGGCAA | |
| TAAAAAGACA GAATAAAACG CACGGTGTTG GGTCGTTTGT TCATAAACGC GGGGTTCGGT | |
| CCCAGGGCTG GCACTCTGTC GATACCCCAC CGAGACCCCA TTGGGGCCAA TACGCCCGCG | |
| TTTCTTCCTT TTCCCCACCC CACCCCCCAA GTTCGGGTGA AGGCCCAGGG CTCGCAGCCA | |
| ACGTCGGGGC GGCAGGCCCT GCCATAGCCT CAGGTTACTC ATATATACTT TAGATTGATT | |
| TAAAACTTCA TTTTTAATTT AAAAGGATCT AGGTGAAGAT CCTTTTTGAT AATCTCATGA | |
| CCAAAATCCC TTAACGTGAG TTTTCGTTCC ACTGAGCGTC AGACCCCGTA GAAAAGATCA | |
| AAGGATCTTC TTGAGATCCT TTTTTTCTGC GCGTAATCTG CTGCTTGCAA ACAAAAAAAC | |
| CACCGCTACC AGCGGTGGTT TGTTTGCCGG ATCAAGAGCT ACCAACTCTT TTTCCGAAGG | |
| TAACTGGCTT CAGCAGAGCG CAGATACCAA ATACTGTCCT TCTAGTGTAG CCGTAGTTAG | |
| GCCACCACTT CAAGAACTCT GTAGCACCGC CTACATACCT CGCTCTGCTA ATCCTGTTAC | |
| CAGTGGCTGC TGCCAGTGGC GATAAGTCGT GTCTTACCGG GTTGGACTCA AGACGATAGT | |
| TACCGGATAA GGCGCAGCGG TCGGGCTGAA CGGGGGGTTC GTGCACACAG CCCAGCTTGG | |
| AGCGAACGAC CTACACCGAA CTGAGATACC TACAGCGTGA GCTATGAGAA AGCGCCACGC | |
| TTCCCGAAGG GAGAAAGGCG GACAGGTATC CGGTAAGCGG CAGGGTCGGA ACAGGAGAGC | |
| GCACGAGGGA GCTTCCAGGG GGAAACGCCT GGTATCTTTA TAGTCCTGTC GGGTTTCGCC | |
| ACCTCTGACT TGAGCGTCGA TTTTTGTGAT GCTCGTCAGG GGGGCGGAGC CTATGGAAAA | |
| ACGCCAGCAA CGCGGCCTTT TTACGGTTCC TGGCCTTTTG CTGGCCTTTT GCTCACATGT | |
| TCTTTCCTGC GTTATCCCCT GATTCTGTGG ATAACCGTAT TACCGCCATG CAT | |

Human cytomegalovirus (CMV) immediate early promoter: 1-589;Enhancer region: 59-465;

TATA box: 554-560;Transcription start point: 583;C→G mutation to remove Sac I site: 569.

MCS: 591-671.

EGFP gene: Kozak consensus translation initiation site: 672-682; Start codon (ATG): 679-681;

Stop codon: 1396-1398; Insertion of Val at position 2: 682-684; GFPmut1 chromophore mutations (Phe-64 to Leu; Ser-65 to Thr): 871-876; His-231 to Leu mutation (A→T): 1373.

SV40 early mRNA polyadenylation signal: Polyadenylation signals: 1552-1557 & 1581-1586 mRNA 3′ ends: 1590 & 1602.

f1 single-strand DNA origin: 1649-2104 (packages the noncoding strand of EGFP).

Bacterial promoter expression of Kanr gene: −35 region: 2166-2171; −10 region: 2189-2194;

Transcription start point: 2201.

SV40 origin of replication: 2445-2580.

SV40 early promoter: Enhancer (72-bp tandem repeats): 2278-2349 & 2350-2421; 21-bp repeats: 2425-2445, 2446-2466, & 2468-2488; Early promoter element: 2501-2507; Major transcription start points: 2497, 2535, 2541 & 2546.

Kanamycin/neomycin resistance gene: Neomycin phosphotransferase coding sequences: Start codon (ATG): 2629-2631; stop codon: 3421-3423; G→A mutation to remove Pst I site: 2811; C→A (Arg to Ser) mutation to remove BssH II site: 3157.

Herpes simplex virus (HSV) thymidine kinase (TK) polyadenylation signal; Polyadenylation signals: 3659-3664 & 3672-3677.

pUC plasmid replication origin: 4008-4651.

The EGFP amino acid sequence (SEQ ID NO: 2) as reported in Genbank for cloning vector pEGFP-N1, locus CVU55762 is

| | |
|---|---|
| MVSKGEELFTGVVPILVELDGDVNGHKFSVSGEGEGDATYGKLT | (end of SEQ ID NO:2) |
| LKFICTTGKLPVPWPTLVTTLTYGVQCFSRYPDHMKQHDFFKSAMPEGYVQERTIFFK | |
| DDGNYKTRAEVKFEGDTLVNRIELKGIDFKEDGNILGHKLEYNYNSHNVYIMADKQKN | |
| GIKVNFKIRHNIEDGSVQLADHYQQNTPIGDGPVLLPDNHYLSTQSALSKDPNEKRDH | |
| MVLLEFVTAAGITLGMDELYK. | |

### REFERENCES

Prasher, D. C., et al. (1992) Primary structure of the Aequorea victoria green fluorescent protein. Gene 111:229-233.

Chalfie, M., et al. (1994) Green fluorescent protein as a marker for gene expression. Science 263: 802-805.

Inouye, S. & Tsuji, F. I. (1994) Aequorea green fluorescent protein: Expression of the gene and fluorescent characteristics of the recombinant protein. FEBS Letters 341:277-280.

Cormack, B. P., et al. (1996) FACS-optimized mutants of the green fluorescent protein (GFP). Gene 173:33-38.

Haas, J., et al. (1996) Codon usage limitation in the expression of HIV-1 envelope glycoprotein. Curr. Biol. 6:315-324.

Li, X., et al. (1998) submitted.

Living Colors Destabilized EGFP Vectors (April 1998) CLONTECHniques XIII(2): 16-17.

Gorman, C. (1985) In DNA Cloning: A Practical Approach, Vol. 11., Ed. Glover, D. M. (IRL Press, Oxford, UK), pp. 143-190.

Information Regarding RFP

This information including SEQ ID NO: 3 is based on information from the Clontech web site. Red Fluorescent Protein (also designated “DsRed”) was isolated from the IndoPacific sea anemone relative Discosoma species. It has a red fluorescence. A plasmid base sequence (SEQ ID NO:3) comprising the coding sequence for RFP is:

| | |
|---|---|
| AGCGCCCAAT ACGCAAACCG CCTCTCCCCG CGCGTTGGCC GATTCATTAA TGCAGCTGGC | (end of SEQ ID NO:3) |
| ACGACAGGTT TCCCGACTGG AAAGCGGGCA GTGAGCGCAA CGCAATTAAT GTGAGTTAGC | |
| TCACTCATTA GGCACCCCAG GCTTTACACT TTATGCTTCC GGCTCGTATG TTGTGTGGAA | |
| TTGTGAGCGG ATAACAATTT CACACAGGAA ACAGCTATGA CCATGATTAC GCCAAGCTTG | |
| CATGCCTGCA GGTCGACTCT AGAGGATCCC CGGGTACCGG TCGCCACCAT GAGGTCTTCC | |
| AAGAATGTTA TCAAGGAGTT CATGAGGTTT AAGGTTCGCA TGGAAGGAAC GGTCAATGGG | |
| CACGAGTTTG AAATAGAAGG CGAAGGAGAG GGGAGGCCAT ACGAAGGCCA CAATACCGTA | |
| AAGCTTAAGG TAACCAAGGG GGGACCTTTG CCATTTGCTT GGGATATTTT GTCACCACAA | |
| TTTCAGTATG GAAGCAAGGT ATATGTCAAG CACCCTGCCG ACATACCAGA CTATAAAAAG | |
| CTGTCATTTC CTGAAGGATT TAAATGGGAA AGGGTCATGA ACTTTGAAGA CGGTGGCGTC | |
| GTTACTGTAA CCCAGGATTC CAGTTTGCAG GATGGCTGTT TCATCTACAA GGTCAAGTTC | |
| ATTGGCGTGA ACTTTCCTTC CGATGGACCT GTTATGCAAA AGAAGACAAT GGGCTGGGAA | |
| GCCAGCACTG AGCGTTTGTA TCCTCGTGAT GGCGTGTTGA AAGGAGAGAT TCATAAGGCT | |
| CTGAAGCTGA AAGACGGTGG TCATTACCTA GTTGAATTCA AAAGTATTTA CATGGCAAAG | |
| AAGCCTGTGC AGCTACCAGG GTACTACTAT GTTGACTCCA AACTGGATAT AACAAGCCAC | |
| AACGAAGACT ATACAATCGT TGAGCAGTAT GAAAGAACCG AGGGACGCCA CCATCTGTTC | |
| CTTTAGCGGC CGCGACTCTA GAATTCCAAC TGAGCGCCGG TCGCTACCAT TACCAACTTG | |
| TCTGGTGTCA AAAATAATAG GCCTACTAGT CGGCCGTACG GGCCCTTTCG TCTCGCGCGT | |
| TTCGGTGATG ACGGTGAAAA CCTCTGACAC ATGCAGCTCC CGGAGACGGT CACAGCTTGT | |
| CTGTAAGCGG ATGCCGGGAG CAGACAAGCC CGTCAGGGCG CGTCAGCGGG TGTTGGCGGG | |
| TGTCGGGGCT GGCTTAACTA TGCGGCATCA GAGCAGATTG TACTGAGAGT GCACCATATG | |
| CGGTGTGAAA TACCGCACAG ATGCGTAAGG AGAAAATACC GCATCAGGCG GCCTTAAGGG | |
| CCTCGTGATA CGCCTATTTT TATAGGTTAA TGTCATGATA ATAATGGTTT CTTAGACGTC | |
| AGGTGGCACT TTTCGGGGAA ATGTGCGCGG AACCCCTATT TGTTTATTTT TCTAAATACA | |
| TTCAAATATG TATCCGCTCA TGAGACAATA ACCCTGATAA ATGCTTCAAT AATATTGAAA | |
| AAGGAAGAGT ATGAGTATTC AACATTTCCG TGTCGCCCTT ATTCCCTTTT TTGCGGCATT | |
| TTGCCTTCCT GTTTTTGCTC ACCCAGAAAC GCTGGTGAAA GTAAAAGATG CTGAAGATCA | |
| GTTGGGTGCA CGAGTGGGTT ACATCGAACT GGATCTCAAC AGCCGTAAGA TCCTTGAGAG | |
| TTTTCGCCCC GAAGAACGTT TTCCAATGAT GAGCACTTTT AAAGTTCTGC TATGTGGCGC | |
| GGTATTATCC CGTATTGACG CCGGGCAAGA GCAACTCGGT CGCCGCATAC ACTATTCTCA | |
| GAATGACTTG GTTGAGTACT CACCAGTCAC AGAAAAGCAT CTTACGGATG GCATGACAGT | |
| AAGAGAATTA TGCAGTGCTG CCATAACCAT GAGTGATAAC ACTGCGGCCA ACTTACTTCT | |
| GACAACGATC GGAGGACCGA AGGAGCTAAC CGCTTTTTTG CACAACATGG GGGATCATGT | |
| AACTCGCCTT GATCGTTGGG AACCGGAGCT GAATGAAGCC ATACCAAACG ACGAGCGTGA | |
| CACCACGATG CCTGTAGCAA TGGCAACAAC GTTGCGCAAA CTATTAACTG GCGAACTACT | |
| TACTCTAGCT TCCCGGCAAC AATTAATAGA CTGGATGGAG GCGGATAAAG TTGCAGGACC | |
| ACTTCTGCGC TCGGCCCTTC CGGCTGGCTG GTTTATTGCT GATAAATCTG GAGCCGGTGA | |
| GCGTGGGTCT CGCGGTATCA TTGCAGCACT GGGGCCAGAT GGTAAGCCCT CCCGTATCGT | |
| AGTTATCTAC ACGACGGGGA GTCAGGCAAC TATGGATGAA CGAAATAGAC AGATCGCTGA | |
| GATAGGTGCC TCACTGATTA AGCATTGGTA ACTGTCAGAC CAAGTTTACT CATATATACT | |
| TTAGATTGAT TTAAAACTTC ATTTTTAATT TAAAAGGATC TAGGTGAAGA TCCTTTTTCA | |
| TAATCTCATG ACCAAAATCC CTTAACCTGA GTTTTCGTTC CACTGAGCGT CAGACCCCGT | |
| AGAAAAGATC AAAGGATCTT CTTGAGATCC TTTTTTTCTG CGCGTAATCT GCTGCTTGCA | |
| AACAAAAAAA CCACCGCTAC CAGCGGTGGT TTGTTTGCCG GATCAAGAGC TACCAACTCT | |
| TTTTCCGAAG GTAACTGGCT TCAGCAGAGC GCAGATACCA AATACTGTCC TTCTAGTGTA | |
| GCCGTAGTTA GGCCACCACT TCAAGAACTC TGTAGCACCG CCTACATACC TCGCTCTGCT | |
| AATCCTGTTA CCAGTGGCTG CTGCCAGTGG CGATAAGTCG TGTCTTACCG GGTTGGACTC | |
| AAGACGATAG TTACCGGATA AGGCGCAGCG GTCGGGCTGA ACGGGGGGTT CGTGCACACA | |
| GCCCAGCTTG GAGCGAACGA CCTACACCGA ACTGAGATAC CTACAGCGTG AGCTATGAGA | |
| AAGCGCCACG CTTCCCGAAG GGAGAAAGGC GGACAGGTAT CCGGTAAGCG GCAGGGTCGG | |
| AACAGGAGAG CGCACGAGGG AGCTTCCAGG GGGAAACGCC TGGTATCTTT ATAGTCCTGT | |
| CGGGTTTCGC CACCTCTGAC TTGAGCGTCG ATTTTTGTGA TGCTCGTCAG GGGGGCGGAG | |
| CCTATGGAAA AACGCCAGCA ACGCGGCCTT TTTACGGTTC CTGGCCTTTT GCTGGCCTTT | |
| TGCTCACATG TTCTTTCCTG CGTTATCCCC TGATTCTGTG GATAACCGTA TTACCGCCTT | |
| TGAGTGAGCT GATACCGCTC GCCGCAGCCG AACGACCGAG CGCAGCGAGT CAGTGAGCGA | |
| GGAAGCGGAA G | |

lac Promoter: 95-178: CAP binding site: 111-124; −35 region: 143-148; −10 region: 167-172:

Transcription start point: 179; lac operator: 179-199.

lacZ-DsRed fusion protein expressed in E. col: Ribosome binding site: 206-209; Start codon (ATG): 217-219; Stop codon 964-966.

5′ Multiple Cloning Site: 234-281.

Discosoma sp. Red Fluorescent Protein (DsRed) gene: Kozak consensus translation initiation site: 282-292; Start codon (ATG): 289-291; Stop codon: 964-966.

3′ Multiple cloning site: 968-1067.

Ampicillin resistance gene: Promoter −35 region: 1441-1446; −10 region: 1464-;

Transcription start point: 1476; Ribosome binding site: 1499-1503; β-lactamase coding sequences (Start codon (ATG): 1511-1513; Stop codon: 2369-2371; β-lactamase signal peptide: 1511-1579; β-lactamase mature protein: 1580-2368).

pUC plasmid replication origin: 2519-3162.

### REFERENCES

Matz, M. V., et al. (1999) Nature Biotech. 17:969 973.

Kozak, M. (1987) Nucleic Acids Res. 15:8125 8148.

Information Regarding ECFP

The following information, including SEQ ID NO:4 was obtained from the Clontech web site:

Restriction Map and Multiple Cloning Site of pECFP: The Xba I sites in the 5′ and 3′ MCSs can be used to excise the ECFP gene.

The vector sequence file has been compiled from information in the sequence database, published literature, and other sources, together with partial sequences obtained by CLONTECH. This vector has not been completely sequenced.

Description: pECFP encodes an enhanced cyan fluorescent variant of the Aequorea victoria green fluorescent protein gene (GFP). The ECFP gene contains six amino acid substitutions. The Tyr-66 to Trp substitution gives ECFP fluorescence excitation (major peak at 433 nm and a minor peak at 453 nm) and emission (major peak at 475 nm and a minor peak at 501 nm) similar to other cyan emission variants. The other five substitutions (Phe-64 to Leu; Ser-65 to Thr; Asn-146 to Ile; Met-153 to Thr; and Val-163 to Ala) enhance the brightness and solubility of the protein.

In addition to the chromophore mutations, ECFP contains >190 silent mutations that create an open reading frame comprised almost entirely of preferred human codons. Upstream sequences flanking ECFP have been converted to a Kozak consensus translation initiation site.

The ECFP gene is flanked at the 5′ and 3′ ends by the two MCSs of the pUC19 derivative pPD16.43. The ECFP coding sequence can be excised from the vector or amplified by PCR. In E. coli, ECFP is expressed from the lac promoter as a fusion with several additional amino acids, including the first five amino acids of the LacZ protein. If one excises the ECFP coding sequence using a restriction site in the 5′ MCS, the resulting fragment will encode the native (i.e., nonfusion) ECFP protein. The pUC19 backbone provides a high-copy-number origin of replication and an ampicillin resistance gene for propagation and selection in E. coli.

Location of Features:

lac promoter: 95-178; CAP binding site: 111-124; −35 region: 143-148; −10 region: 167-172; Transcription start point: 179; lac operator: 179-199.

lacZ-ECFP Fusion Protein Expressed in E. coli:

Ribosome binding site: 206-209

Start codon (ATG): 217-219; stop codon: 1006-1008.

5′ Multiple Cloning Site: 234-281.

Enhanced Cyan Fluorescent Protein (ECFP) Gene:

Kozak consensus translation initiation site: 282-292.

Start codon (ATG): 289-291; stop codon: 1006-1008.

Insertion of Val at position 2: 292-294.

ECFP mutations (Phe-64 to Leu, Ser-65 to Thr, and Tyr-66 to Trp): 481-489;

Asn-146 to Ile: 727-729; Met-153 to Thr: 748-750; Val-163 to Ala: 778-780

His-231 to Leu mutation (A→T): 983.

3′ Multiple Cloning Site: 1010-1109.

Ampicillin Resistance Gene:

Promoter: −35 region: 1485-1490; −10 region: 1508-1513;

Transcription start point: 1520;

Ribosome binding site: 1543-1547;

beta-lactamase coding sequences:

Start codon (ATG): 1555-1557;

stop codon: 2413-2415;

b-lactamase signal peptide: 1555-1623;

b-lactamase mature protein: 1624-2412;

pUC Plasmid Replication Origin: 2563-3206.

Primer Locations:

EGFP-N Sequencing Primer (#6479-1): 355-334.

EGFP-C Sequencing Primer (#6478-1): 942-963.

Propagation in E. coli:

Recommended host strain: JM109

Selectable marker: plasmid confers resistance to ampicillin (100 μg/ml) to E. coli hosts

E. coli replication origin: pUC

Copy number: ˜500

Plasmid incompatibility group: pMB1/ColE1.

### REFERENCES

Heim, R. & Tsien, R. Y. (1996) Engineering green fluorescent protein for improved brightness, longer wavelengths and fluorescence resonance energy transfer. Curr. Biol. 6:178-182.

Mitra, R. D., et al. (1996) Fluorescence resonance energy transferbetween blue-emitting and red-shifted excitation derivatives of the green fluorescent protein. Gene 173:13-17.

Heim, R., et al. (1994) Wavelength mutations and posttranslational autoxidation of green fluorescent protein. Proc. Natl. Acad. Sci. USA 91:12501-12504.

Cormack, B. P., et al. (1996) FACS-optimized mutants of the green fluorescent protein (GFP). Gene 173:33-38.

Yang, T. T., et al. (1996) Optimized codon usage and chromophore mutations provide enhanced sensitivity with the green fluorescent protein. Nucleic Acids Res. 24:4592-4593.

Haas, J., et al. (1996) Codon usage limitation in the expression of HIV-1 envelope glycoprotein. Curr. Biol. 6(3):315-324.

Kozak, M. (1987) An analysis of 5′-noncoding sequences from 699 vertebrate messenger RNAs. Nucleic Acids Res. 15:8125-8148.

Fire, A., et al. (1990) A modular set of lacZ fusion vectors for studying gene expression in Caenorhabditis elegans. Gene 93:189-198.

pECFP nucleotide sequence from Clontech (SEQ ID NO:4):

| | |
|---|---|
| AGCGCCCAAT ACGCAAACCG CCTCTCCCCG CGCGTTGGCC GATTCATTAA TGCAGCTGGC ACGACAGGTT | (End of SEQ ID NO:4) |
| TCCCGACTGG AAAGCGGGCA GTGAGCGCAA CGCAATTAAT GTGAGTTAGC TCACTCATTA GGCACCCCAG | |
| GCTTTACACT TTATGCTTCC GGCTCGTATG TTGTGTGGAA TTGTGAGCGG ATAACAATTT CACACAGGAA | |
| ACAGCTATGA CCATGATTAC GCCAAGCTTG CATGCCTGCA GGTCGACTCT AGAGGATCCC CGGGTACCGG | |
| TCGCCACCAT GGTGAGCAAG GGCGAGGAGC TGTTCACCGG GGTGGTGCCC ATCCTGGTCG AGCTGGACGG | |
| CGACGTAAAC GGCCACAAGT TCAGCGTGTC CGGCGAGGGC GAGGGCGATG CCACCTACGG CAAGCTGACC | |
| CTGAAGTTCA TCTGCACCAC CGGCAAGCTG CCCGTGCCCT GGCCCACCCT CGTGACCACC CTGACCTGGG | |
| GCGTGCAGTG CTTCAGCCGC TACCCCGACC ACATGAAGCA GCACGACTTC TTCAAGTCCG CCATGCCCGA | |
| AGGCTACGTC CAGGAGCGCA CCATCTTCTT CAAGGACGAC GGCAACTACA AGACCCGCGC CGAGGTGAAG | |
| TTCGAGGGCG ACACCCTGGT GAACCGCATC GAGCTGAAGG GCATCGACTT CAAGGAGGAC GGCAACATCC | |
| TGGGGCACAA GCTGGAGTAC AACTACATCA GCCACAACGT CTATATCACC GCCGACAAGC AGAAGAACGG | |
| CATCAAGGCC AACTTCAAGA TCCGCCACAA CATCGAGGAC GGCAGCGTGC AGCTCGCCGA CCACTACCAG | |
| CAGAACACCC CCATCGGCGA CGGCCCCGTG CTGCTGCCCG ACAACCACTA CCTGAGCACC CAGTCCGCCC | |
| TGAGCAAAGA CCCCAACGAG AAGCGCGATC ACATGGTCCT GCTGGAGTTC GTGACCGCCG CCGGGATCAC | |
| TCTCGGCATG GACGAGCTGT ACAAGTAAAG CGGCCGCGAC TCTAGAATTC CAACTGAGCG CCGGTCGCTA | |
| CCATTACCAA CTTGTCTGGT GTCAAAAATA ATAGGCCTAC TAGTCGGCCG TACGGGCCCT TTCGTCTCGC | |
| GCGTTTCGGT GATGACGGTG AAAACCTCTG ACACATGCAG CTCCCGGAGA CGGTCACAGC TTGTCTGTAA | |
| GCGGATGCCG GGAGCAGACA AGCCCGTCAG GGCGCGTCAG CGGGTGTTGG CGGGTGTCGG GGCTGGCTTA | |
| ACTATGCGGC ATCAGAGCAG ATTGTACTGA GAGTGCACCA TATGCGGTGT GAAATACCGC ACAGATGCGT | |
| AAGGAGAAAA TACCGCATCA GGCGGCCTTA AGGGCCTCGT GATACGCCTA TTTTTATAGG TTAATGTCAT | |
| GATAATAATG GTTTCTTAGA CGTCAGGTGG CACTTTTCGG GGAAATGTGC GCGGAACCCC TATTTGTTTA | |
| TTTTTCTAAA TACATTCAAA TATGTATCCG CTCATGAGAC AATAACCCTG ATAAATGCTT CAATAATATT | |
| GAAAAAGGAA GAGTATGAGT ATTCAACATT TCCGTGTCGC CCTTATTCCC TTTTTTGCGG CATTTTGCCT | |
| TCCTGTTTTT GCTCACCCAG AAACGCTGGT GAAAGTAAAA GATGCTGAAG ATCAGTTGGG TGCACGAGTG | |
| GGTTACATCG AACTGGATCT CAACAGCGGT AAGATCCTTG AGAGTTTTCG CCCCGAAGAA CGTTTTCCAA | |
| TGATGAGCAC TTTTAAAGTT CTGCTATGTG GCGCGGTATT ATCCCGTATT GACGCCGGGC AAGAGCAACT | |
| CGGTCGCCGC ATACACTATT CTCAGAATGA CTTGGTTGAG TACTCACCAG TCACAGAAAA GCATCTTACG | |
| GATGGCATGA CAGTAAGAGA ATTATGCAGT GCTGCCATAA CCATGAGTGA TAACACTGCG GCCAACTTAC | |
| TTCTGACAAC GATCGGAGGA CCGAAGGAGC TAACCGCTTT TTTGCACAAC ATGGGGGATC ATGTAACTCG | |
| CCTTGATCGT TGGGAACCGG AGCTGAATGA AGCCATACCA AACGACGAGC GTGACACCAC GATGCCTGTA | |
| GCAATGGCAA CAACGTTGCG CAAACTATTA ACTGGCGAAC TACTTACTCT AGCTTCCCGG CAACAATTAA | |
| TAGACTGGAT GGAGGCGGAT AAAGTTGCAG GACCACTTCT GCGCTCGGCC CTTCCGGCTG GCTGGTTTAT | |
| TGCTGATAAA TCTGGAGCCG GTGAGCGTGG GTCTCGCGGT ATCATTGCAG CACTGGGGCC AGATGGTAAG | |
| CCCTCCCGTA TCGTAGTTAT CTACACGACG GGGAGTCAGG CAACTATGGA TGAACGAAAT AGACAGATCG | |
| CTGAGATAGG TGCCTCACTG ATTAAGCATT GGTAACTGTC AGACCAAGTT TACTCATATA TACTTTAGAT | |
| TGATTTAAAA CTTCATTTTT AATTTAAAAG GATCTAGGTG AAGATCCTTT TTGATAATCT CATGACCAAA | |
| ATCCCTTAAC GTGAGTTTTC GTTCCACTGA GCGTCAGACC CCGTAGAAAA GATCAAAGGA TCTTCTTGAG | |
| ATCCTTTTTT TCTGCGCGTA ATCTGCTGCT TGCAAACAAA AAAACCACCG CTACCAGCGG TGGTTTGTTT | |
| GCCGGATCAA GAGCTACCAA CTCTTTTTCC GAAGGTAACT GGCTTCAGCA GAGCGCAGAT ACCAAATACT | |
| GTCCTTCTAG TGTAGCCGTA GTTAGGCCAC CACTTCAAGA ACTCTGTAGC ACCGCCTACA TACCTCGCTC | |
| TGCTAATCCT GTTACCAGTG GCTGCTGCCA GTGGCGATAA GTCGTGTCTT ACCGGGTTGG ACTCAAGACG | |
| ATAGTTACCG GATAAGGCGC AGCGGTCGGG CTGAACGGGG GGTTCGTGCA CACAGCCCAG CTTGGAGCGA | |
| ACGACCTACA CCGAACTGAG ATACCTACAG CGTGAGCTAT GAGAAAGCGC CACGCTTCCC GAAGGGAGAA | |
| AGGCGGACAG GTATCCGGTA AGCGGCAGGG TCGGAACAGG AGAGCGCACG AGGGAGCTTC CAGGGGGAAA | |
| CGCCTGGTAT CTTTATAGTC CTGTCGGGTT TCGCCACCTC TGACTTGAGC GTCGATTTTT GTGATGCTCG | |
| TCAGGGGGGC GGAGCCTATG GAAAAACGCC AGCAACGCGG CCTTTTTACG GTTCCTGGCC TTTTGCTGGC | |
| CTTTTGCTCA CATGTTCTTT CCTGCGTTAT CCCCTGATTC TGTGGATAAC CGTATTACCG CCTTTGAGTG | |
| AGCTGATACC GCTCGCCGCA GCCGAACGAC CGAGCGCAGC GAGTCAGTGA GCGAGGAAGC GGAAG | |

### EXAMPLES

The following examples are intended to illustrate the invention, not limit it.

### Example 1

Effect of P-Glycoprotein GFP Fusion Protein Expression on Drug Accumulation

How much of the decreased accumulation of drugs and fluorescent dyes can be attributed to Pgp expression alone? Our approach was to transiently transfect cells with a fusion of Pgp and green or cyan fluorescent protein. This produced a diverse population of cells, ranging from those that expressed large amounts of Pgp to those that failed to express the protein at all. The activity of Pgp was then quantified in individual cells that had been exposed to the same treatments, but differed substantially in their levels of Pgp. Two assays were used to examine the effects of the Pgp-fluorescent protein on chemotherapeutic drugs. First, we examined the cellular accumulation of structurally divergent MDR fluorescent dyes including those that are constitutively positively charged, weakly basic or uncharged. Second, we measured the cellular activity of microtubule disrupting chemotherapeutics by their effect on microtubules. Thus the activity of Pgp was quantitatively studied as a function of its levels of expression in individual cells.

To assure that a correctly folded, full-length fusion protein was produced in the transfected cells, we used immunoblotting and immunofluorescence. The PgpGFP fusion protein was detected as a ˜200 kD band by either an anti-Pgp antibody (clone F4) or anti-GFP antibody. Wild type Pgp appeared as a ˜170 kD band (data not shown). Immunofluorescence of three epitope-specific anti-Pgp antibodies (clones F4, 4E3, UIC2) co-localized with GFP fluorescence (data not shown).

Weak Base Chemotherapeutic Drugs:

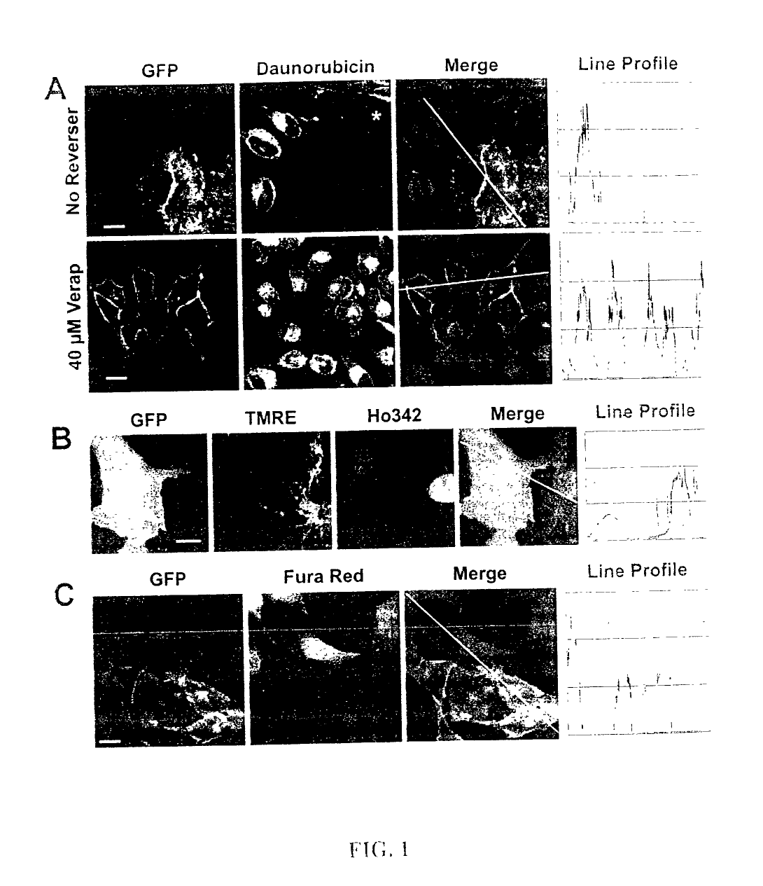

Daunorubicin is a weak base with one protonatable nitrogen at physiological pKa (Altan et al., 1998). Cells expressing PgpGFP accumulated dramatically less daunorubicin (FIG. 1A). Note that in this field, there were three cells that did not express Pgp (left), one that expressed a high level of protein (bottom right) and one that produced a small amount (top right, asterisk). The level of Pgp expression was inversely correlated with daunorubicin accumulation. For example, the daunomycin fluorescence was more than 100-fold lower in the cell that expressed a high level of Pgp compared to its Pgp negative neighbor. Similarly, the level of Pgp expression was inversely related to the cellular accumulation of mitoxantrone, a clinically important anthracenedione chemotherapeutic that is also a weak base but that is significantly different in structure from daunorubicin (data not shown). The MDR reversers verapamil (40 μM, FIG. 1A) and cyclosporin A (10 uM, data not shown), completely reversed the effect of PgpGFP expression on accumulation of daunorubicin. The UIC2 anti-Pgp antibody, at 10 μ/kg/ml, partially reversed the effect of PgpGFP expression (data not shown).

Positively Charged Dyes:

We tested the effects of expressing PgpGFP on two MDR compounds that have constitutive positive charges, the DNA stain Hoechst 33342 (Ho342) and the mitochondrial dye tetramethylrhodamine methyl ester (TMRE). (TMRE is similar to the well known MDR dye rhodamine 123 whose fluorescence overlaps GFP.) In cells expressing PgpGFP the TMRE fluorescence was virtually undetectable and the Ho342 fluorescence was 3-fold lower (FIG. 1B), consistent with previous reports on the specificity of Pgp (Lizard et al., 1995a). The accumulation of both Ho324 and TMRE in the cells expressing PgpGFP was increased by the MDR-reversers verapamil (40 μM) and cyclosporin A (10 μM) (data not shown). Staining with both TMRE and Ho342 serves as a good control for cell fitness. In unhealthy cells, the loss of mitochondrial membrane potential limits accumulation of TMRE while the degradation of the membrane permits large amounts of Ho342 to enter (Sun et al., 1992;Lizard et al., 1995b).

Acetoxymethyl Esters:

The effects of PgpGFP expression was tested on a number of uncharged acetoxymethyl (AM) esters implicated as MDR substrates. AM esters of many hydrophilic indicator dyes are used to facilitate cellular loading (Homolya et al., 1993). Within the cell, esterases cleave AM groups, trapping the dye inside the cell. Expression of PgpCFP or PgpGFP substantially reduced the cellular accumulation of several AM esters such as Fura Red (FIG. 1C). Adding verapamil (40 μM) increased Fura Red accumulation in PgpGFP expressing cells (data not shown). The effects of PgpGFP on reducing the accumulation of BCECF, calcein, and Fura-2 (Table 1) correlates with the ability of each AM ester dye to stimulate the ATPase activity of Pgp (Homolya et al., 1993).

Microtubule-Disrupting Drugs:

We used a functional assay to quantify the effect of PgpGFP expression on the cytosolic activity of chemotherapeutics. Certain chemotherapeutics, such as colchicine, vincristine and nocodazole, depolymerize microtubules. The state of a cell's microtubules after being treated with these drugs is a measurement of the cellular concentration of the drug. Thus, we examined the microtubules using immunofluorescence against b-tubulin after drug treatment.

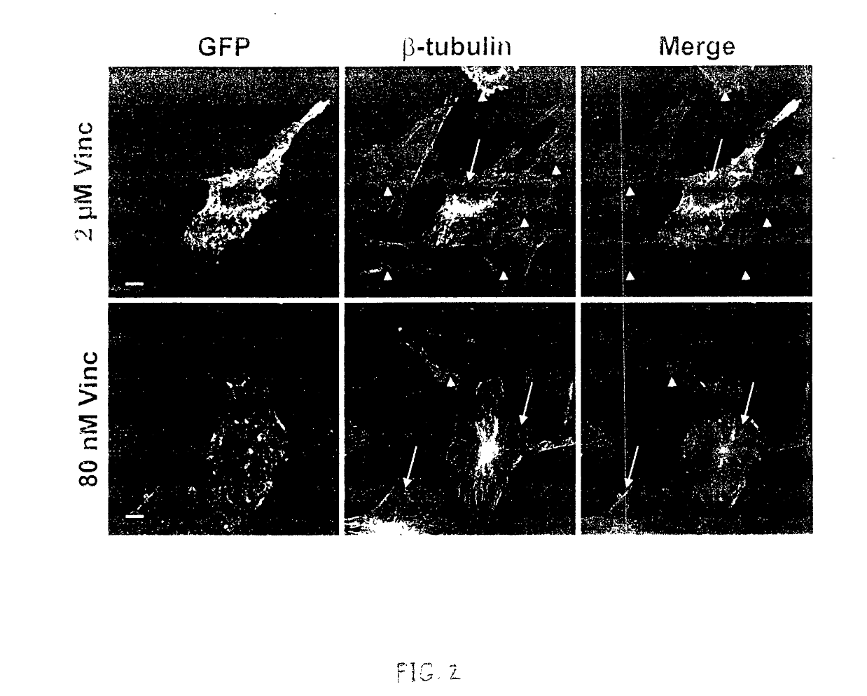

Cells expressing PgpGFP maintained microtubules in 80 nM vincristine while non-expressing cells in the same field did not (FIG. 2B). Cells expressing high levels of PgpGFP had intact microtubules even in 2 mM vincristine (FIG. 2A). Thus, expression of PgpGFP correlates with a greater than 25-fold decrease in vincristine accumulation. Expression of PgpGFP also decreased the sensitivity of cells to colchicine, but had no effect on sensitivity to nocodazole (Table 2).

Expression-Activity Profile of PgpGFP:

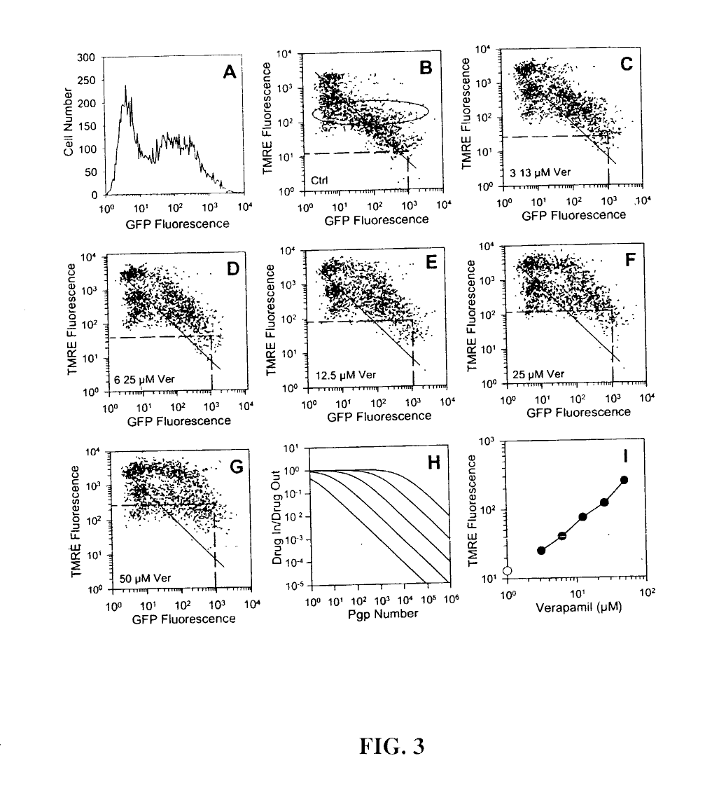

Various mechanisms have been proposed to account for the effects of Pgp on drug accumulation in the cell (Wadkins and Roepe, 1997;Stein, 1997;Eytan and Kuchel, 1999). Each has a different characteristic dependence of drug accumulation on Pgp expression level. We used FACS to quantify PgpGFP expression and TMRE accumulation over a wide dynamic range. Approximately 50% of HeLa cells expressed PgpGFP and the expression level varied over 100-fold (FIG. 3A). In cells expressing the highest level of PgpGFP, the average TMRE accumulation was 100-fold less than in non-expressing cells. A dot plot of TMRE fluorescence versus GFP fluorescence shows an inverse linear relationship on a logarithmic scale with a slope close to −1, and a linear fit of slope −1 is shown as the solid line (FIG. 3B). Hence, it appears that expression of PgpGFP has an inverse linear relationship to TMRE accumulation. This relationship is consistent only with a model in which Pgp mediates active efflux itself, without cooperativity between either enzyme or substrate (see below).

For an idealized cell with a plasma membrane efflux pump, where a single substrates interacts with a single enzyme, the steady state ratio of cellular drug concentration (Dᵢₙ) to external concentration (Dₒᵤₜ) follows the following equation: Dᵢₙ/Dₒᵤₜ≈1/(1+X) where X=N.C/(Kₘ.P.S) (see Appendix). Here, N is the number of pumps, C is the catalytic constant, P is the drug permeability and S plasma membrane surface area. When X is less than 1, the cellular drug concentration approaches the external drug concentration and increasing X does little. When X is greater than 1, the ratio approaches 1/X, an inversely linear relationship. We modeled this equation using the following approximate constants: P=10⁻⁵ cm/s, S=5000 μm, Kₘ=10 μM. FIG. 3H shows a plot Dᵢₙ/Dₒᵤₜ as a function of the number of pump molecules per cell. The five plots, from left to right, assume catalytic constants of 10, 1, 0.1, 0.01, and 0.001 drug molecules pumped per Pgp per second.

The model predicts that an inhibitor should shift the TMRE accumulation to PgpGFP relationship to the right, analogous to decreasing the catalytic constant in FIG. 3H. This was tested by co-incubating cells with TMRE and 3.13 μM, 6.25 μM, 12.5 μM, 25 μM or 50 μM verapamil (FIGS. 3C-G respectively). The solid lines on these figures is the fit from FIG. 3B as a reference. Indeed, as the concentration of verapamil increased, the curve shifted right. To quantify the effect of verapamil, we estimated the average TMRE fluorescence of cells showing GFP fluorescence of 10³ at different verapamil concentrations using the dash lines in FIGS. 3B-G. The plot of TMRE fluorescence versus verapamil concentration shows an approximate linear relationship (FIG. 31), as expected from a specific inhibitor and lack of cooperativity between inhibitor molecules (see Appendix). Our data estimate the Kᵢ of verapamil to be 3 μM, in full agreement with published data (Lan et al., 1996). Thus, the FACS data shows that Pgp mediates active drug efflux, and that verapamil is a specific inhibitor.

Effect of PgpGFP expression on cellular pH: MDR cells have been shown to have higher cytosolic pH (Keizer and Joenje, 1989;Simon et al., 1994). Since such higher pH results in decreased concentration of weak base drugs this could be the consequence of selection in chemotherapeutics rather than a specific effect of expressing Pgp. However, stable transfection of Pgp has been reported to raise cytosolic pH (Thiebaut et al., 1990) even without chemotherapeutic drug selection (Hoffman et al., 1996).

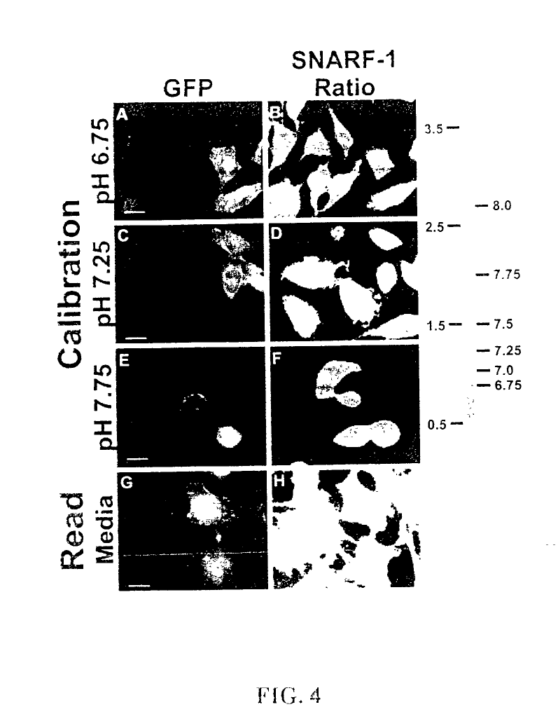

We examined the effect of PgpGFP expression on cytosolic pH using SNARF-1. FIGS. 4A-F show calibration at three different pH. As expected, the ratio increased with increasing pH in an exponential manner. PgpGFP expression did not affect the ratio of the calibration images. Measurement of cellular pH of cells in medium showed that both PgpGFP expressing and non-expressing cells had a cytosolic pH of approximately 7.2 (FIGS. 4G, H). Thus, Pgp expression has no effect on cellular pH.

Discussion:

The goal of our study was to determine if Pgp expression in the absence of any selection was sufficient to produce multidrug resistance and, if so, to understand the mechanism of Pgp. To this end, we devised a technique that allowed the levels of Pgp expression to be directly compared with cellular accumulation of chemotherapeutics. We designed a novel method that took advantage of GFP, a protein that has revolutionized cell biology by permitting the study of localization and movement of proteins in living cells. We extended the range of application of GFP to the study of biochemical processes in living cells. Traditionally, enzymes are studied in vitro, away from their natural cellular environment. Enzyme analysis in living cells is hampered by two constraints. First, intracellular enzyme concentration usually varies within a narrow range and, second, enzyme concentration and intracellular localization cannot be easily measured. The use of transient transfection addresses the first problem since it generates a large range of expression levels. Using GFP fusion proteins addresses the second problem. A previous approach to in vivo enzyme analysis also employed transient transfection of the enzyme. In that study, enzyme activity was detected with a fluorescent substrate. The amount of enzyme was measured by subsequent immunoquantification (Morita et al., 1995). By using a GFP fusion protein allowed us to measure simultaneously the quantity, localization and kinetic activity of an enzyme in living cells.

Expression of PgpGFP resulted in dramatically decreased accumulation of many diverse compounds, including the non-charged AM-esters, the weak base drug daunorubicin, and the constitutively charged dyes TMRE and Ho342. Decreased accumulation was also inferred from the decreased sensitivities to microtubule depolymerizing drugs vincristine and colchicine. This effect of PgpGFP expression was inhibited by an antibody against Pgp and MDR reversers.

FACS analysis showed that PgpGFP expression and TMRE accumulation had an inverse linear relationship, implying that PgpGFP mediates active efflux. Furthermore, this data implies that the TMRE extrusion is a bimolecular reaction: a single molecule of TMRE is pumped at a time and a single Pgp unit (monomer or stable multimer) is the catalytic unit. FACS analysis further showed that the concentration of verapamil and level of TMRE accumulation in PgpGFP expressing cells were linearly related, suggesting that verapamil is an inhibitor and that there is no interaction between pairs of verapamil molecules.

Tumor cells are known to be genetically unstable and that exposure to mutagenic compounds invariably results in many mechanisms of drug resistance. Which combinations of these mechanisms are clinically relevant has yet to be established. The use of GFP fusions permits the study of any one individual protein independently from other drug resistance phenomena.

### Appendix to Example 1

We use the pump/leak model (Stein, 1997;Eytan and Kuchel, 1999). The drug influx rate is leak in and the drug efflux rate is the leak out plus the pump rate out:

Influx=P.S.Dₒᵤₜ

Efflux=P.S.Dᵢₙ+N.C.Dᵢₙ/(Dᵢₙ+Kₘ)

where the constants are defined in the text. To further simplify our analysis, we assume that Dᵢₙ is small compared to Kₘ, which is safe since we used a TMRE concentration of 50 nM. The pump rate then approximates N.C.Dᵢₙ/Kₘ. The equilibrium concentration must satisfy the equation Influx=Efflux, giving

Dᵢₙ/Dₒᵤₜ>>1/(1+X); X=N.C/(Kₘ.P.S).

A non-competitive inhibitor decreases the apparent C according to the following formula:

ti Cₐₚₚ/C=(Kᵢ/Kᵢ+I)

where I is the inhibitor concentration. Thus, the apparent C is halved when I=Kᵢ. When I>>Kᵢ, Cₐₚₚ approaches an inverse linear relationship with I. For a competitive inhibitor, the apparent Kₘ is increased such that

Kₘ/Kₘₐₚₚ=Kᵢ/Kᵢ+I

and the same analysis shows that when I=Kᵢ, Kₘₐₚₚ is doubled and Kₘₐₚₚ is linearly related to I when I>>Kᵢ.

### Example 2

### Hydrolysis of p-nitrophenol-α-D-maltosehexoside by MalS

In analogy to Example 1, the hydrolysis of p-nitrophenol-α-D-maltosehexoside by MalS so as to form maltohexose can be studied by creating a MalS-GFP plasmid construct and infecting E. coli cells. The assay is performed as described by Freundlieb et. al. (1988) with minor modifications. Cultures are grown at 37° C. in M9 with 0.2% dextrose, 0.2% cas-amino acids, 50 mg/ml Amp and 50 mg/ml Cm. When cultures reach an OD595˜0.6, IPTG is added to various concentrations, and cultures are grown for an additional 2 hours. The cells are centrifuged for 10 min. at 3,000 g at room temperature, washed in fresh M9 medium without IPTG, and resuspended in fresh M9 medium to an OD595=2.0 (1×10⁹ cells/ml). For cell permeabilization, 1:20 volume of 0.1% SDS and chloroform are added, the samples are vortexed vigorously, and allowed to stand for 10 minutes. The chromogenic substrate p-nitrophenyl-alpha-D-maltohexaoside (PG6) is added to the tubes to a final concentration of 2 mM or as desired. (S. Freundlieb, U. Ehmann, and W. Boos. Facilitated diffusion of p-nitrophenyl-alpha-D-maltohexaoside through the outer membrane of Escherichia coli. Characterization of LamB as a specific and saturable channel for maltooligosaccharides. J. Biol. Chem. 263 (1):314-320, 1988.)

### REFERENCES

Abraham, E. H., A. G. Prat, L. Gerweck, T. Seneveratne, R. J. Arceci, R. Kramer, and G. Guidotti. 1993. The multidrug resistance (mdr1) gene product functions as an ATP channel. Proc. Natl. Acad. Sci. USA 90:312-316.

Altan, N., Y. Chen, M. Schindler, and S. M. Simon. 1998. Defective acidification in human breast tumor cells and implications for chemotherapy. J. Exp. Med. 187:1583-1598.

Altan, N., Y. Chen, M. Schindler, and S. M. Simon. 1999. Tamoxifen inhibits acidification in cells independent of the estrogen receptor. Proc. Natl. Acad. Sci. USA 96:4432-4437.

Bech-Hansen, N. T., J. E. Till, and V. Ling. 1976. Pleiotropic phenotype of colchicine-resistant CHO cells: cross-resistance and collateral sensitivity. J. Cell. Physiol. 88:23-31.

Biedler, J. L., H. Riehm, R. H. Peterson, and B. A. Spengler. 1975. Membrane-mediated drug resistance and phenotypic reversion to normal growth behavior of Chinese hamster cells. J. Natl. Canc. Inst. 55:671-680.

Chalfie, M., Tu, Y., Euskirchen, G., Ward, W. W. and Prasher, D. 1994. Green fluorescent protein as a marker for gene expression. Science 263, 802-805.

Deffie, A. M., T. Alam, C. Seneviratne, S. W. Beenken, J. K. Batra, T. C. Shea, W. D. Henner, and G. J. Goldenberg. 1988. Multifactorial resistance to adriamycin: relationship of DNA repair, glutathione transferase activity, drug efflux, and P-glycoprotein in cloned cell lines of adriamycin-sensitive and -resistant P388 leukemia. Cancer Res. 48:3595-3602.

Eytan, G. D. and P. W. Kuchel. 1999. Mechanism of action of P-glycoprotein in relation to passive membrane permeation. Int. Rev. Cytol. 190:175-250.

Gill, D. R., S. C. Hyde, C. F. Higgins, M. A. Valverde, G. M. Mintenig, and F. V. Sepulveda. 1992. Separation of drug transport and chloride channel functions of the human multidrug resistance P-glycoprotein. Cell 71:23-32.

Gottesman, M. M. and I. Pastan. 1993. Biochemistry of multidrug resistance mediated by the multidrug transporter. Annu. Rev. Biochem. 62:385-427.

Hoffman, M. M., L. Y. Wei, and P. D. Roepe. 1996. Are altered pHi and membrane potential in hu MDR 1 transfectants sufficient to cause MDR protein-mediated multidrug resistance?. J. Gen. Physiol. 108:295-313.

Homolya, L., Z. Holló, U. A. Germann, I. Pastan, M. M. Gottesman, and B. Sarkadi. 1993. Fluorescent cellular indicators are extruded by the multidrug resistance protein. J. Biol. Chem. 268:21493-21496.

Kartner, N., J. R. Riordan, and V. Ling. 1983. Cell surface P-glycoprotein associated with multidrug resistance in mammalian cell lines. Science 221:1285-1288.

Keizer, H. G. and H. Joenje. 1989. Increased cytosolic pH in multi drug-resistant human lung tumor cells: effect of verapamil. J. Natl. Cancer Inst. 81:706-709.

Kunkel, T. A. 1985. Rapid and efficient site-specific mutagenesis without phenotypic selection. Proc. Natl. Acad. Sci. U.S. A 82:488-492.

Lan, L. B., S. Ayesh, E. Lyubimov, I. Pashinsky, and W. D. Stein. 1996. Kinetic parameters for reversal of the multidrug pump as measured for drag accumulation and cell killing. Cancer Chemotherapy & Pharmacology 38:181-190.

Ling, V. 1997. Multidrug resistance: Molecular mechanisms and clinical relevance. Cancer Chemother. Pharmacol. 40:S3-S8.

Lizard, G., S. Foumel, L. Genestier, N. Dhedin, C. Chaput, M. Flacher, M. Mutin, G. Panaye, and J. P. Revillard. 1995b. Kinetics of plasma membrane and mitochondrial alterations in cells undergoing apoptosis. Cytometry 21:275-283.

Lizard, G., M. Maynadie, P. Roignot, S. Lizard-Nacol, and M. F. Poupon. 1995a. Evaluation of multidrug resistant phenotype by flow cytometry with monoclonal antibodies and functional tests. Bull. Cancer 82:211-217.

Morita, I., W. L. Smith, D. L. DeWitt, and M. Schindler. 1995. Expression-activity profiles of cells transfected with prostaglandin endoperoxide H synthase measured by quantitative fluorescence microscopy. Biochemistry 34:7194-7199.

Robinson, L. J., W. K. Roberts, T. T. Ling, D. Lamming, S. S. Stemberg, and P. D. Roepe. 1997. Human MDR 1 protein overexpression delays the apoptotic cascade in Chinese hamster ovary fibroblasts. Biochemistry 36:11169-11178.

Roepe, P. D., L. Yong Wei, J. Cruz, and D. Carlson. 1993. Lower electrical membrane potential and altered pH, homeostasis in multidrug-resistant (MDR) cells: Further characterization of a series of MDR cell lines expressing different levels of P-glycoprotein. Biochemistry 32:11042-11056.

Schindler, M., S. Grabski, E. Hoff, and S. M. Simon. 1996. Defective pH regulation of acidic compartments in human breast cancer cells (MCF-7) is normalized in adriamycin-resistant cells (MCF-7adr). Biochemistry 35:2811-2817.

Simon, S. M., D. Roy, and M. Schindler. 1994. Intracellular pH and the control of multidrug resistance. Proc. Natl. Acad. Sci. USA 91:1128-1132.

Simon, S. M. and M. Schindler. 1994. Cell biological mechanisms of multidrug resistance in tumors. Proc. Natl. Acad. Sci. USA 91:3497-3504.

Stein, W. D. 1997. Kinetics of the multidrug transporter (P-glycoprotein) and its reversal. Physiol. Rev. 77:545-590.

Sun, X. M., R. T. Snowden, D. N. Skilleter, D. Dinsdale, M. G. Ormerod, and G. M. Cohen. 1992. A flow-cytometric method for the separation and quantitation of normal and apoptotic thymocytes. Anal. Biochem. 204:351-356.

Thiebaut, F., S. J. Currier, J. Whitaker, R. P. Haugland, M. M. Gottesman, I. Pastan, and M. C. Willingham. 1990. Activity of the multidrug transporter results in alkalinization of the cytosol: measurement of cytosolic pH by microinjection of a pH-sensitive dye. Journal of Histochemistry & Cytochemistry 38:685-690.

Tsien, R. Y. 1998. The green fluorescent protein. Annu Rev Biochem 67:509-544.

Wadkins, R. M. and P. D. Roepe. 1997. Biophysical aspects of P-glycoprotein-mediated multidrug resistance. International Review of Cytology 171:121-165.

Tables

TABLE 1
The effect of PgpGFP or PgpCFP
expression on accumulation of AM esters

| Dye | Effects on fluorescence | |
|---|---|---|
| BCECF AM (CFP) | 3-10 | Fold |
| Calcein AM (CFP) | >10 | Fold |
| FURA-2 AM (GFP) | >10 | Fold |
| FURA Red AM (GFP) | >10 | Fold |
| SNAFL-1 diacetate (CFP) | >10 | Fold |
| SNAFL calcein AM (CFP) | >10 | Fold |
| SNARE-1 AM (GFP) | <33 | Fold |

| | |
|---|---|
| SNARE calcein AM (GFP) | None |

TABLE 2
The effect of high level PgpGFP
expression on drug mediated microtubule depolymerization

| Drug | Fold resistance |
|---|---|
| Colchicine | 5-25 fold |
| Nocodazole | None |
| Vincristine | 25-125 fold |

What is claimed is:

1. A quantification process for simultaneously quantifying in situ the relationship between an enzyme and its substrate, said process comprising the steps of:
1) creating a population of cells that synthesize said enzyme, such that all cells in the population do not contain equal amounts of said enzyme, and wherein the enzyme is tagged with a fluorescent agent;
2) incubating the population of cells in the presence of the substrate;
3) simultaneously, by optical means, quantifying in each member of the population the intracellular concentration of the enzyme and its enzymatic activity.

2. A process of claim 1 wherein the enzyme is fluorescently tagged.

3. A process of claim 2 wherein the enzyme is fluorescently tagged by fusing it to a fluorescent protein so that the enzyme and the fluorescent protein are part of the same protein.

4. A process of claim 1 wherein the substrate has a color or is fluorescent; which color or fluorescence is distinguishable from the emitted fluorescence of the tagged enzyme;

5. A process of claim 4 wherein the substrate is fluorescent and its emitted fluorescence emission is distinguishable from the emitted fluorescence of the tagged enzyme.

6. A process of claim 1 wherein a product of the enzyme's action on the substrate has a color or is fluorescent, which color or fluorescence is distinguishable from the emitted fluorescence of the tagged enzyme.

7. A process of claim 1 wherein the population of cells is created by transfecting it with a nucleic acid construct that, upon infecting a cell, causes expression of the fluorescently tagged enzyme,

8. A process of claim 7 wherein the construct comprises a base sequence coding for said enzyme.

9. A process of claim 1 wherein quantification of fluorescence comprises analysis of individual cells.

10. A process of claim 9 wherein step (3) of the process is performed a plurality of times.
